(19) United States
(12) Patent Application Publication
(10) Pub. No.: US 2023/0016709 A1
(43) Pub. Date: Jan. 19, 2023

(54) USE OF PROTEASE SERINE 21 (PRSS21) ANTIGEN TESTING IN THE DIAGNOSIS AND TREATMENT OF ACUTE MYELOID LEUKEMIA

(71) Applicant: St. Jude Children's Research Hospital, Memphis, TN (US)

(72) Inventors: Stephen Gottschalk, Memphis, TN (US); Patrick Schreiner, Memphis, TN (US); Yiping Fan, Memphis, TN (US); Nikhil Hebbar, Memphis, TN (US); Myreya Paulina Velasquez, Memphis, TN (US)

(73) Assignee: St. Jude Children's Research Hospital, Memphis, TN (US)

(21) Appl. No.: 17/779,841

(22) PCT Filed: Nov. 24, 2020
(86) PCT No.: PCT/IB2020/061103

§ 371 Date: May 25, 2022

Related U.S. Application Data
(60) Provisional application No. 62/939,924, filed on Nov. 25, 2019.

(51) Int. Cl. C12Q 1/6886; A61P 35/02; A61K 35/17

(52) U.S. Cl. CPC C12Q 1/6886; A61P 35/02; A61K 35/17; C12Q 2600/118; C12Q 2600/106; C12Q 2600/158

(57) ABSTRACT

Methods for detection, diagnosis, prognosis, theragnosis, and targeted therapy of a PRSS21-overexpressing condition (e.g., cancer), in particular, PRSS21-overexpressing acute myeloid leukemia of the AMKL subtype.

### FIELD

The present disclosure relates to the technical fields of cancer biomarkers, immunology, and medicine.

### BACKGROUND

Acute myeloid leukemia (AML) is the second most common form of childhood leukemia and the most common second malignancy in children who were initially treated for other cancers. AML varies greatly in nature and prognosis depending on their WHO classification. This category uses morphology, immunophenotype and genetic and clinical finding to assign a category. Acute megakaryoblastic leukemia (AMKL) is a particularly unfavorable subtype for which improved diagnostic, prognostic, and therapeutic interventions are needed to improve the prognosis of patients, particularly pediatric patients.

Targeted agents are routinely used to treat many cancers and have improved outcomes for patients with solid tumors and hematological malignancies. Recently, targeted therapies have become available for AML, representing the first therapeutic advances for AML in decades. However, effective immunotherapy targets in pediatric AML, particularly for the unfavorable AML subtypes, are still not available. Thus, there is an unmet need in the art to identify candidate immunotherapy targets/biomarkers (e.g., Antibody and CAR-T targets) in pediatric B-cell acute lymphoblastic leukemia (B-ALL) and AML, particularly for treating unfavorable subtypes such as AMKL. Further, there is a need for precise biomarkers that can identify a subject as a candidate for such anti-cancer immunotherapies.

### SUMMARY

Provided herein are methods that are useful in the detection, diagnosis, monitoring, prognosis, theragnosis, and/or treatment of a condition (e.g., cancer) associated with an elevated expression of protease serine 21 (PRSS21).

In one aspect, provided herein is a method for diagnosing and/or prognosing a cancer associated with PRSS21 expression that involves the steps of measuring the level of PRSS21 in a biological sample by contacting the biological sample with a PRSS21 detection agent, and measuring the absence, presence or expression level of an expression product of PRSS21, wherein increased level of the PRSS21 expression product compared to control indicates that the patient has cancer associated with PRSS21 expression.

In some embodiments, the cancer is selected from the group that includes a leukemia, B-cell acute lymphoblastic leukemia (B-ALL), acute myeloid leukemia (AML) pediatric B-ALL, pediatric AML, AML of the AMKL subtype, and a solid tumor.

In some embodiments, the biological sample is selected from the group that includes fluids (e.g., blood, plasma, serum, bone marrow aspirate, bronchoalveolar lavage (BAL), and cerebrospinal fluid (CSF)), tissues, cell samples, organs, biopsies, and tumor samples.

In some embodiments, the expression product is an RNA, an mRNA, a protein, or a fragment thereof.

In some embodiments, the detection agent is a nucleic acid (e.g., DNA or RNA probe) or an antibody.

In some embodiments, the expression is measured by array hybridization, direct hybridization of RNA, digital quantitation of transcript levels, quantitative PCR, quantitative sequencing, northern blot analysis, mass spectrometry, ELISA, flow cytometry, immunohistochemistry, radioimmunoassay, western blot, or immunoprecipitation.

In another aspect, provided herein is a method for identifying a subject as a candidate for an anti-cancer immunotherapy by determining an expression level of PRSS21 in a biological sample (e.g., fluids (e.g., blood, plasma, serum, bone marrow aspirate, BAL, and CSF), tissues, cell samples, organs, biopsies, or tumor samples) from the subject, wherein an increased expression level of PRSS21 in the biological sample from the subject compared to a reference expression level in a non-cancerous sample is indicative of the subject being a candidate for anti-PRSS21 immunotherapy.

In some embodiments of the aforementioned aspect, the cancer is selected from the group that includes a leukemia, B-ALL, AML, pediatric B-ALL, pediatric AML, AML of the AMKL subtype, and a solid tumor.

Another aspect provides a method for identifying a subject as a candidate for an anti-cancer immunotherapy including a chimeric antigen receptor (CAR)-T cell therapy, by determining an expression level of PRSS21 in a biological sample (e.g., fluids (e.g., blood, plasma, serum, bone marrow aspirate, BAL, and CSF), tissues, cell samples, organs, biopsies, or tumor samples) from the subject, wherein an increased expression level of PRSS21 in the biological sample from the subject compared to a reference expression level in a non-cancerous sample is indicative of the subject being a candidate for anti-PRSS21 immunotherapy.

Another aspect provides a method for identifying a subject suffering from AML or a solid tumor as a candidate for an anti-cancer immunotherapy including a CAR-T cell therapy, by determining an expression level of PRSS21 in a biological sample (e.g., fluids (e.g., blood, plasma, serum, bone marrow aspirate, BAL, and CSF), tissues, cell samples, organs, biopsies, or tumor samples) from the subject wherein an increased expression level of PRSS21 in the biological sample from the subject compared to a reference expression level in a non-cancerous sample is indicative of the subject being a candidate for anti-PRSS21 immunotherapy.

Another aspect provides a method of treating a subject having a cancer associated with an elevated expression of PRSS21, by administering to the subject an effective amount of an immunotherapeutic composition that includes an anti-PRSS21 antibody or an immune effector cell targeted to PRSS21.

Another aspect provides a method of treating a subject suffering AML or a solid tumor associated with an elevated expression of PRSS21, by administering to the subject an effective amount of an immunotherapeutic composition that includes an anti-PRSS21 antibody or an immune effector cell targeted to PRSS21.

In yet another aspect is provided a method for targeted anti-cancer immunotherapy in a subject suffering from AML (e.g., AMKL subtype) or a solid tumor associated with an elevated expression of PRSS21, wherein the anti-cancer immunotherapy includes administration of a therapeutically effective amount of a CAR-T cell.

Another aspect provides a method for treating a subject with an anti-PRSS21 immunotherapy, wherein the subject is suffering from a PRSS21-associated disorder, the method including the steps of measuring the level of PRSS21 in a biological sample (obtained from the patient) by contacting the biological sample with a PRSS21 detection agent, and measuring the absence, presence, or expression level of an expression product (e.g., RNA, an mRNA, a protein, or a fragment thereof) of PRSS21, and wherein if an increased level of the PRSS21 expression product is observed, then administering a therapeutically effectively amount of an immunotherapeutic agent to the patient.

In some embodiments of the aforementioned aspect, the PRSS21-associated disorder is a cancer, such as a cancer selected from the group that includes a leukemia, B-ALL, AML, pediatric B-ALL, pediatric AML, AML of the AMKL subtype, and a solid tumor. In particular embodiments, the cancer is AML of the AMKL subtype.

In some embodiments of the above aspect, PRSS21 level is measured by array hybridization, direct hybridization of RNA, digital quantitation of transcript levels, quantitative PCR, quantitative sequencing, northern blot analysis, mass spectrometry, ELISA, flow cytometry, immunohistochemistry, radioimmunoassay, western blot, or immunoprecipitation.

In some embodiments of the above aspect, the immunotherapeutic agent includes a CAR-T.

### BRIEF DESCRIPTION OF THE DRAWINGS

FIG. 1: Comparison of Gene Expression Distributions Across Data Cohorts. Violin plots of gene expression value distributions across cohorts using non-negative, non-zero (A) log₂ expression values, (B) log₂ expression values with CUMULPERC transformation, and (C) log₂ expression values with CUMULPERC transformation then quantile normalization18. Dotted red lines indicate the median of values associated with absent calls and the dotted green lines indicate the median of values associated with present calls in the microarray data.

FIG. 2: Gene Status Prediction Based Upon Transformed Microarray Hybridization Values. ROC curves were used to assess the performance of the logistic regression model in (A) a ten-fold cross-validation, as well as in an (B) external microarray dataset.

FIG. 3: Workflow for CAR-T Cell Therapy Target Identification. The workflow shows the steps by which CAR-T targets were identified in pediatric patients with (A) B-ALL and (B) AMKL. The numbers between steps indicate the number of CAR-T candidates still under consideration after each filter.

FIG. 4: Utilizing Gene Status Predictions to Estimate CAR-T Target Efficacy in Individual Patients. Boxplots of transformed gene expression values showing all patient samples, only patients in which the gene status was predicted as “Present,” and only patients in which the gene status was predicted as “Absent.” (A) CD19 and (B) CD22 are shown as positive controls for effective CAR-T in B-ALL. Top algorithm hits in AMKL patients are shown for (C) adult AML clinical trial target, CD33, and (D) novel CD family AML CAR-T target, CD42b. (E) PRSS21 and (F) PRAME are shown to represent potential CAR-T targets that are not in the CD gene family.

### DETAILED DESCRIPTION

### Overview

In the United States, the number of estimated acute myeloid leukemia (AML) cases in 2018 was 19,520 and 10,670 of those patients are expected die from AML. About 730 individuals under the age of 20 are diagnosed with AML each year in the United States. That makes AML the second most common form of childhood leukemia and the most common second malignancy in children that were initially treated for other cancers. Pediatric AML survival is currently reported between 65-70%, which is much lower than reported in other pediatric leukemia like acute lymphoblastic leukemia (ALL). (Hunger, S. P, et al., “Children's Oncology Group's 2013 blueprint for research: acute lymphoblastic leukemia,” Pediatr. Blood Cancer. 2013, 60(6):957-963.

Acute myeloid leukemia (AML), also called acute nonlymphocytic, granulocytic, myelocytic, myeloblastic, or myeloid leukemia, is a disease in which cancer cells develop in the blood and bone marrow. The cancer develops from two main types of immature white blood cells that normally develop into mature granulocytes or monocytes. The result is a malignancy characterized by the accumulation in blood and bone marrow of abnormal hematopoietic progenitors and disruption of normal production of erythroid, myeloid, and/or megakaryocytic cell lines.

AML varies greatly in nature and prognosis based upon the World Health Organization (WHO) classification. The WHO classification of AML incorporate clinical, prognostic, morphologic, immunophenotypic, and genetic data to divide AML into the following groups (Arber D. A. et al., Blood 127:2391-2405 (2016)):

(i) AML with certain genetic abnormalities (gene or chromosome changes) (e.g., AML with a translocation between chromosomes 8 and 21 [t(8;21)], AML with a translocation or inversion in chromosome 16 [t(16;16) or inv(16)], APL with the PML-RARA fusion gene; AML with a translocation between chromosomes 9 and 11 [t(9;11)], AML with a translocation between chromosomes 6 and 9 [t(6:9)], AML with a translocation or inversion in chromosome 3 [t(3;3) or inv(3)], AML (megakaryoblastic) with a translocation between chromosomes 1 and 22 [t(1:22)], AML with the BCR-ABL1 (BCR-ABL) fusion gene (provisional entity), AML with mutated NPM1 gene, AML with biallelic mutations of the CEBPA gene (that is, mutations in both copies of the gene), and AML with mutated RUNX1 gene (provisional entity));

(ii) AML with myelodysplasia-related changes;

(iii) AML related to previous chemotherapy or radiation (or therapy-related myeloid neoplasms); and

(iv) AML not otherwise specified (AML, NOS) (e.g., AML with minimal differentiation (FAB M0), AML without maturation (FAB M1), AML with maturation (FAB M2), Acute myelomonocytic leukemia (FAB M4), Acute monoblastic/monocytic leukemia (FAB M5), Pure erythroid leukemia (FAB M6), Acute megakaryoblastic leukemia (AMKL, FAB M7), Acute basophilic leukemia, and Acute panmyelosis with fibrosis).

Recently developed targeted therapies for AML include, for example, FLT3 inhibitors (e.g., Midostaurin (Rydapt), Gilteritinib (Xospata)), IDH inhibitors (e.g., Ivosidenib (Tibsovo), Enasidenib (Idhifa)), Gemtuzumab ozogamicin (Mylotarg), BCL-2 inhibitors (Venetoclax (Venclexta), and Hedgehog pathway inhibitors (e.g., Glasdegib (Daurismo)). While some of these drugs are useful in treating certain people with AML, not all AML patients benefit from these therapies.

Immunotherapy, such as immune checkpoint blockade and chimeric antigen receptor T cell (CAR-T) therapy, has demonstrated clinical efficacy in directing the specific recognition and activation of response by the immune system without the need for HLA typing. (Pardoll, D. M., “The blockade of immune checkpoints in cancer immunotherapy,” Nat. Rev. Cancer. 2012, 12(4):252). Targeted immunotherapy has the potential to make a major impact in the treatment of conditions that are difficult to treat like metastatic cancers, multiple sclerosis, Crohn's disease, and other autoimmune disorders. (Ellebrecht, C. T., et al., “Reengineering chimeric antigen receptor T cells for targeted therapy of autoimmune disease,” Science, 2016; 353(6295):179-184) For example, CD-19 targeted CAR-T therapy has proven effective in the treatment of B-cell malignancies. (Maude, S. L., et al., “CD19-targeted chimeric antigen receptor T cell therapy for acute lymphoblastic leukemia,” Blood. 2015, 125(26):4017-4023). Effective CAR-T targets in pediatric AML are still under investigation.

Public data availability has become a requisite in scientific research. As a result, publicly available datasets are continually increasing in quantity, diversity, and focus. However, data heterogeneity makes meaningful comparisons difficult between samples or data cohorts derived from different protocols, assays, and instruments. For example, the tumor samples may be measured by RNA-seq, but the normal controls may be measured by microarray or RNA-seq with a different protocol or sequencing center. The massive collection of microarray data is still a very useful resource, even though RNA-seq is now the main platform for measuring gene expression. Microarray data measures relative expression, while RNA-seq measures quantitative read counts, so a typical method is not directly applicable to normalize RNA-seq and microarray data together.

Making reliable and meaningful comparisons using publicly available data is critical for effective use of the plethora of data that is publicly available to drive novel research and confirm the consistency of research findings across data cohorts. To that end, Applicant has developed a method by which heterogeneous data—data derived from multiple sources, protocols, instruments and experiment types—is transformed and compared to predict effective immunotherapy targets (e.g., CAR-T targets) in pediatric B-ALL and AML. The methodology described herein to identify candidate CAR targets can also be applied more generally in cancers with gene expression data in tumor relative to normal tissue.

### PRSS21

PRSS21 serine protease 21 (also known as eosinophil serine protease 1 (ESP1), testisin (TEST1)) is a membrane-type serine protease, which is a member of the trypsin family of serine proteases. The encoded protein is predicted to be active on peptide linkages involving the carboxyl group of lysine or arginine. The encoded protein localizes to the cytoplasm and the plasma membrane of premeiotic testicular germ cells, and may be involved in progression of testicular tumors of germ cell origin. Alternatively spliced transcript variants encoding multiple isoforms have been observed for this gene, including, for example, variants described by NCBI Reference Sequence Numbers NM_006799.4, NM_144957.3, NM_144956.3, NM_001270452.2, NR_073012.2, XM_017022875.1, XM_017022878.1, XM_017022876.1, and XM_017022877.1.

In certain aspects, the present disclosure provides a method that is useful in the detection, diagnosis, monitoring, prognosis, theragnosis, and/or treatment of a condition (e.g., cancer) associated with an elevated expression of protease serine 21 (PRSS21). In certain embodiments, the disclosure provides a biomarker for identifying a subject having a condition (e.g., a cancer) associated with an elevated expression of protease serine 21 (PRSS21). Exemplary cancers include, but are not limited to leukemias, B-cell acute lymphoblastic leukemia (B-ALL), Acute myeloid leukemia (AML) pediatric B-ALL, pediatric AML, AMKL, solid tumors expressing PRSS21, cervical squamous cell carcinoma and endocervical adenocarcinoma, esophageal carcinoma, lung squamous cell carcinoma, ovarian serous cystadenocarcinoma, pancreatic adenocarcinoma, stomach adenocarcinoma, thymoma, uterine carcinosarcoma, uterine carcinosarcoma, and testicular germ cell tumors.

As used herein, the term “prognosis” means a prediction of the probable course and outcome of the cancer (e.g., AML), including the following: (a) patient prognosis in the absence of therapy (e.g., chemotherapy or radiation therapy; (b) prognosis of patient response to treatment (chemotherapy, radiation therapy) (c) predicted optimal course of treatment for the patient; (d) prognosis for patient relapse after treatment, for example, by prognosis of minimal residual disease (such patients have high risk of relapse), and (e) patient life expectancy.

The term “theragnosis” is used herein as defined in the art to refer to strategies that combine simultaneous diagnosis and therapy. It associates both a diagnostic test that identifies patients most likely to be helped or harmed by a new medication, and targeted drug therapy based on the test results. Theragnostic approaches allow for use of new targeted (tailored) therapies (e.g., CAR) with adequate benefit/risk to patients, and to optimize drug selection. Further, theragnostic approaches can eliminate the unnecessary treatment of patients for whom therapy is not appropriate, resulting in significant drug cost savings for the healthcare system. Such tailored approaches are particularly useful for patients suffering from treatment resistant cancer types, or those known to have less favorable/unfavorable treatment outcomes (e.g., AMKL). In some aspects, the present disclosure provides a method for the administration of a specific immunotherapy (e.g., CAR therapy) involving the detection of an increased level of PRSS21 in a biological sample from a subject.

In some aspects, the present disclosure relates to methods for detecting or measuring PRSS21 expression in a biological sample obtained from a patient. In certain embodiments, the method comprises measuring the level of PRSS21 in a biological sample by contacting the biological sample with a PRSS21 detection agent (e.g., nucleic acid probe, anti-PRSS21 antibody or a fragment thereof), and detecting the absence, presence or expression level of the expression product (mRNA or protein) of PRSS21 (e.g., binding between PRSS21 and an anti-PRSS21 antibody or a fragment thereof), wherein an increased level of PRSS21 compared to control indicates that the patient has a cancer associated with PRSS21 expression. The term “biological sample” means any biological sample derived from a patient, e.g., a sample which contains nucleic acids or proteins. Examples of such samples include, but are not limited to fluids (e.g., blood, plasma, serum, bone marrow aspirate, bronchoalveolar lavage (BAL), cerebrospinal fluid (CSF)), tissues, cell samples, organs, biopsies, tumor samples. Cancer cells from a metastase or cancer cells obtained from blood as circulating tumor cells may also be used. The biological sample may be treated prior to its use, e.g. in order to render nucleic acids or proteins available. Techniques of cell lysis, concentration or dilution of nucleic acids or proteins, are known to persons of skill in the art. The disclosure further provides an article of manufacture (e.g., diagnostic kits) useful for detecting PRSS21 expression in a biological sample obtained from a patient.

More generally the presence and/or level(s) of PRSS21 may be measured qualitatively or quantitatively using any of a number of techniques available to the person of ordinary skill in the art for protein or nucleic acid analysis, e.g., direct physical measurements (e.g., mass spectrometry), binding assays (e.g., immunoassays, agglutination assays, and immunochromatographic assays), polymerase chain reaction (PCR, RT-PCR; RT-qPCR) technology, branched oligonucleotide technology, Northern blot technology, oligonucleotide hybridization technology (e.g., direct hybridization, array hybridization) and in situ hybridization technology. The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

As used herein, “increased expression of PRSS21”, “elevated expression of PRSS21”, “increased level of PRSS21”, and “elevated level of PRSS21” refer to any statistically significant increase in the expression level of the PRSS21 gene or protein level of the PRSS21 protein. Expression level can be measured by any method known in the art for measuring the gene expression. Likewise, the level of protein can be measured by any method known in the art for measuring protein content. An “increased” or “elevated” level could be any statistically significant increase including an increase of 5%, 10%, 15%, 20%, 25%, 30%, 35%, 40%, 45%, 50%, 55%, 60%, 65%, 70%, 75%, 80%, 85%, 90%, 95%, or more when compared to an appropriate control level of PRSS21 or expression level of PRSS21. As used herein a control can be a sample of normal (i.e., non-cancerous) cells of the same type as the test sample or a cell sample known to be free of PRSS21 overexpressing cells.

In certain embodiments, the methods can comprise combining, for example, a cell-containing a test sample with an anti-PRSS21 antibody, assaying the test sample for antibody binding to cells in the test sample and comparing the level of antibody binding in the test sample to the level of antibody binding in a control sample of cells. A suitable control is, e.g., a sample of normal cells of the same type as the test sample or a cell sample known to be free of PRSS21 overexpressing cells. A level of PRSS21 binding higher than that of such a control sample would be indicative of the test sample containing cells that overexpress PRSS21. Alternatively, the control may be a sample of cells known to contain cells that overexpress PRSS21. In such a case, a level of PRSS21 antibody binding in the test sample that is similar to, or in excess of, that of the control sample would be indicative of the test sample containing cells that overexpress PRSS21.

In certain embodiments, expression of PRSS21 may be assayed by immunohistochemistry (IHC) assays or variants thereof (e.g., fluorescent, chromogenic, standard ABC, standard LSAB, etc.), immunocytochemistry or variants thereof (e.g., direct, indirect, fluorescent, chromogenic, etc.). In exemplary embodiments, paraffin embedded tissue sections from a tumor biopsy may be subjected to the IHC assay and accorded a PRSS21 protein staining intensity criteria as follows: Score 0 no staining is observed or membrane staining is observed in less than 10% of tumor cells. Score 1+ a faint/barely perceptible membrane staining is detected in more than 10% of the tumor cells. The cells are only stained in part of their membrane. Score 2+ a weak to moderate complete membrane staining is observed in more than 10% of the tumor cells. Score 3+ a moderate to strong complete membrane staining is observed in more than 10% of the tumor cells. Those tumors with 0 or 1+ scores for PRSS21 expression may be characterized as not overexpressing PRSS21, whereas those tumors with 2+ or 3+ scores may be characterized as overexpressing PRSS21. In other embodiments, scoring may be measurement of percent of positively stained PRSS21 cells in a biological sample, e.g., a tumor sample. In this regard patients exhibiting a certain percentage of positively stained cells in an IHC sample when interrogated with an anti-PRSS21 antibody would be considered PRSS21+ and can be selected for the methods described herein (e.g., CAR therapy). In such embodiments tumor samples exhibiting greater than 10%, greater than 20%, greater than 30%, greater than 40% or greater than 50% positive cell staining may be classified as PRSS21+ when measured as percent positive cells. In other embodiments tumor samples exhibiting greater than 60%, greater than 70%, greater than 80%, greater than 90% or greater than 95% positive cell staining may be classified as PRSS21+ when measured as percent positive.

In certain embodiments, fluorescence in situ hybridization (FISH) assays may be carried out on formalin-fixed, paraffin-embedded tumor tissue to determine the extent (if any) of PRSS21 overexpression in the tumor. PRSS21 overexpression or amplification may be evaluated using an in vivo diagnostic assay, e.g. by administering a molecule (such as an antibody of this invention) which binds PRSS21 and which is labeled with a detectable label (e.g. a radioactive isotope or a fluorescent label) and externally scanning the patient for localization of the label. In other embodiments, the expression of PRSS21 in a biological sample can be measured using flow cytometry comprising fluorescent antibody staining.

A sample suspected of containing cells expressing or overexpressing PRSS21 is combined with suitable anti-PRSS21 antibodies (or fragments thereof) under conditions suitable for the specific binding of the antibodies to PRSS21. Binding and/or internalizing the PRSS21 antibodies of this invention is indicative of the cells expressing PRSS21. The level of binding may be determined and compared to a suitable control, wherein an elevated level of bound PRSS21 as compared to the control is indicative of PRSS21 overexpression. The sample suspected of containing cells overexpressing PRSS21 may be a cancer cell sample, for example a sample of an AMKL subtype cancer. A serum sample from a subject may also be assayed for levels of PRSS21 by combining a serum sample from a subject with an anti-PRSS21 antibody, determining the level of PRSS21 bound to the antibody and comparing the level to a control, wherein an elevated level of PRSS21 in the serum of the patient as compared to a control is indicative of overexpression of PRSS21 by cells in the patient. The subject may have a cancer such as e.g., AMKL subtype cancer.

In certain embodiments, in situ hybridization is used to detect or monitor PRSS21. In situ hybridization technology or ISH is well known to those of skill in the art. Briefly, cells are fixed and detectable probes which contain a specific nucleotide sequence are added to the fixed cells. If the cells contain complementary nucleotide sequences, the probes, which can be detected, will hybridize to them. Using the sequence information set forth herein, probes can be designed to identify cells that express genotypic PRSS21. Probes preferably hybridize to a nucleotide sequence that correspond to PRSS21, including fragments thereof. Hybridization conditions can be routinely optimized to minimize background signal by non-fully complementary hybridization though preferably the probes are preferably fully complementary to PRSS21. In selected embodiments the probes are labeled with fluorescent dye attached to the probes that is readily detectable by standard fluorescent methodology.

Compatible in vivo theragnostics or diagnostic assays may comprise art-recognized imaging or monitoring techniques such as magnetic resonance imaging, computerized tomography (e.g. CAT scan), positron tomography (e.g., PET scan) radiography, ultrasound, etc., as would be known by those skilled in the art.

In some aspects, the present disclosure provides a method of identifying a subject as a candidate for an anti-cancer immunotherapy comprising determining an expression level of PRSS21 in the cancer, wherein an increased expression level of PRSS21 compared to a reference expression level in a non-cancerous sample is indicative of the subject being a candidate for anti-PRSS21 immunotherapy. A subject who is identified as having an increased expression level of PRSS21 will receive a different therapy than a subject who does not have an increased expression level of PRSS21.

In some aspects, the present disclosure provides a method of identifying a subject as a candidate for an anti-cancer immunotherapy comprising a chimeric antigen receptor (CAR)-T cell therapy, comprising determining an expression level of PRSS21 in the cancer wherein an increased expression level of PRSS21 compared to a reference expression level in a non-cancerous sample is indicative of the subject being a candidate for anti-PRSS21 immunotherapy.

In some aspects, the present disclosure provides a method of identifying a subject suffering from acute myelogenic leukemia (AML) or a solid tumor as a candidate for an anti-cancer immunotherapy comprising a chimeric antigen receptor (CAR)-T cell therapy, comprising determining an expression level of PRSS21 in the cancer wherein an increased expression level of PRSS21 compared to a reference expression level in a non-cancerous sample is indicative of the subject being a candidate for anti-PRSS21 immunotherapy A subject who is identified as having an increased expression level of PRSS21 will receive a different therapy than a subject who does not have an increased expression level of PRSS21.

### Articles of Manufacture

In certain embodiments, the disclosure provides a packaged article comprising one or more containers or receptacles, e.g., an article of manufacture, such as an assay and/or diagnostic kit, comprising any of the PRSS21 detection agent (e.g., nucleic acid probes, antibodies) optionally with a label(s) and/or with instructions for use. Such label(s) include(s) ingredients, amounts or dosages, and/or indications. Such instructions include directing or promoting, including advertising, use of said article of manufacture. In certain other embodiments, the packaged article contains a detectable amount of a PRSS21 detection agent, with or without an associated reporter molecule and optionally one or more additional agents for the detection, quantitation and/or visualization of cancerous cells. When the components of the kit are provided in one or more liquid solutions, the liquid solution can be non-aqueous, though typically an aqueous solution is preferred, with a sterile aqueous solution being particularly preferred. The formulation in the kit can also be provided as dried powder(s) or in lyophilized form that can be reconstituted upon addition of an appropriate liquid. The liquid used for reconstitution can be contained in a separate container. Such liquids can comprise sterile, pharmaceutically acceptable buffer(s) or other diluent(s) such as bacteriostatic water for injection, phosphate-buffered saline, Ringer's solution or dextrose solution. Where the kit comprises a PRSS21 detection agent in combination with additional diagnostic markers or agents, the solution may be pre-mixed, either in a molar equivalent combination, or with one component in excess of the other. Alternatively, the PRSS21 detection agent and any optional diagnostic agent or other agent can be maintained separately within distinct containers prior to use.

In certain embodiments the aforementioned kits comprising compositions of the invention will comprise a label, marker, package insert, bar code and/or reader indicating that the kit contents may be used for the detection, prognosis, diagnosis, and/or theragnosis of cancer. In other embodiments the kit may comprise a label, marker, package insert, bar code and/or reader indicating that the kit contents may be administered in accordance with a certain protocol or regimen to diagnose a subject suffering from cancer. In a particularly preferred aspect the label, marker, package insert, bar code and/or reader indicates that the kit contents may be used for the detection, prognosis, diagnosis, and/or theragnosis of a hematologic malignancy (e.g., AML) or provide a protocol or regimen for diagnosis of the same. In other particularly preferred aspects the label, marker, package insert, bar code and/or reader indicates that the kit contents may be used for the detection, prognosis, diagnosis, and/or theragnosis of a solid tumor.

Suitable containers include, for example, bottles, vials, syringes, etc. The containers can be formed from a variety of materials such as glass or pharmaceutically compatible plastics. In certain embodiments the container(s) can comprise a sterile access port, for example, the container may be an intravenous solution bag or a vial having a stopper that can be pierced by a hypodermic injection needle.

In some embodiments the kit can contain a means by which to dispense the PRSS21 detection agent and any optional components, e.g., one or more needles or syringes (pre-filled or empty), a dropper, pipette, or other such like apparatus, from which the formulation may be injected or introduced onto a detection surface (e.g., a slide or a microtiter plate), or into a subject for collecting a tumor sample, or applied to a diseased area of the body. The kits of the invention will also typically include a means for containing the vials, or such like, and other components in close confinement for commercial sale, such as, e.g., blow-molded plastic containers into which the desired vials and other apparatus are placed and retained.

In certain embodiments, the invention relates to a method of manufacturing an article of manufacture comprising any of the PRSS21 detection agents or compositions described herein, packaging the same to obtain an article of manufacture and instructing, directing or promoting the use of the article of manufacture for any of the uses described herein. Such instructing, directing or promoting includes advertising.

In certain embodiments, any of the assays, including diagnostic kits or any article of manufacture comprising the PRSS21 detection agents or compositions described herein may be used as a companion diagnostic.

In other embodiments, any of the aforementioned methods and articles of manufacture may be used in conjunction with, or comprise, diagnostic or therapeutic compositions and/or devices useful in the prevention or treatment of a PRSS21 expressing cancer. For example, in some embodiments the methods and articles of manufacture may be combined with certain diagnostic devices or instruments that may be used to detect, monitor, quantify or profile cells or marker compounds involved in the etiology or manifestation of proliferative disorders. In yet other embodiments the compositions and/or devices may be used to detect, monitor and/or quantify circulating tumor cells either in vivo or in vitro.

In certain aspects, the disclosure provides a method of treating a subject having cancer comprising administering an effective amount of an anti-cancer immunotherapy to the subject, said subject having been determined to have a cancer comprising an elevated expression level of protease serine 21 (PRSS21) compared to a reference expression level in a non-cancerous sample. In certain embodiments, the cancer comprises a leukemia or a solid tumor. In certain aspects, the disclosure provides a method of administering an effective amount of an immunotherapy/immunotherapeutic agent specific for a condition (e.g., a cancer) expressing PRSS21. In particular embodiments, the disclosure provides a method of administering an effective amount of an immunotherapy/immunotherapeutic agent specific for acute myeloid leukemia (AML) expressing PRSS21. In particular embodiments, the AML comprises the AMKL subtype. In some aspects, the disclosure provides a method of treating a subject suffering from acute myelogenic leukemia (AML), the method comprising administering an effective amount of an anti-cancer immunotherapy to the subject, wherein the AML is associated with an elevated expression level of protease serine 21 (PRSS21) compared to a reference expression level in a non-cancerous sample. In certain embodiments, the AML comprises an AMKL subtype. In certain aspects, the disclosure provides a method of treating a subject suffering from acute myeloid leukemia (AML), the method comprising administering an effective amount of an anti-cancer immunotherapy to the subject, wherein the AML is associated with an elevated expression level of protease serine 21 (PRSS21) compared to a reference expression level, and wherein said expression level is measured by an assay comprising array hybridization, direct hybridization of RNA, digital quantitation of transcript levels, quantitative PCR, quantitative sequencing, northern blot analysis, etc.; protein level can be measured by mass spectrometry, ELISA, flow cytometry, immunohistochemistry, radioimmunoassay, western blot assay, or immunoprecipitation. In certain aspects, the disclosure provides a method of treating a subject having a cancer associated with an elevated expression of protease serine 21 (PRSS21), comprising administering to the subject an effective amount of an immunotherapeutic composition comprising an anti-PRSS21 antibody or an immune effector cell targeted to PRSS21. In some embodiments, the cancer comprises AML, or a solid tumor. In any of the aforementioned methods, the anti-cancer immunotherapy can comprise an antibody (e.g., an anti-PRSS21 monoclonal antibody, a CAR-T cell, or a combination thereof, including a combination with any conventional AML therapy).

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. A subject or mammal is successfully “treated” for a PRSS21-expressing cancer if, after receiving a therapeutic amount of an anti-PRSS21 antibody according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the anti-PRSS21 antibody may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR).

The terms “effective amount” or “therapeutically effective amount” refer to an amount of a therapeutic agent (e.g., an antibody, CAR, or a drug) effective to “treat” a disease or disorder in a subject or mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. To the extent the drug may prevent growth and/or kill existing cancer cells, it may be cytostatic and/or cytotoxic.

In certain aspects, the disclosure provides a method for treating a subject with an anti-PRSS21 immunotherapy, wherein the subject is suffering from a PRSS21-associated disorder (e.g., cancer), the method comprising the steps of: measuring the level of PRSS21 in a biological sample (obtained from the patient) by contacting the biological sample with a PRSS21 detection agent, and measuring the absence, presence, or expression level of the expression product of PRSS21; and wherein if an increased level of PRSS21 is observed, then administer a therapeutically effectively amount of a specific immunotherapy (e.g., CAR) to the patient. In particular embodiments, the cancer is an AML, and more specifically AMKL subtype.

In particular embodiments, the patient identified by the methods disclosed herein can be treated with FLT3 inhibitors (e.g., Midostaurin (Rydapt), Gilteritinib (Xospata)), IDH inhibitors (e.g., Ivosidenib (Tibsovo), Enasidenib (Idhifa)), Gemtuzumab ozogamicin (Mylotarg), BCL-2 inhibitors (Venetoclax (Venclexta), and Hedgehog pathway inhibitors (e.g., Glasdegib (Daurismo)).

### EXAMPLES

### Example 1. Unifying Heterogeneous Expression Data to Predict Targets of CAR-T Therapy

CAR-T therapy has demonstrated the capacity to serve as a molecular mechanism to target and kill cancer cells. The efficacy of CAR-T cell therapy relies heavily on the identification of an antigen that has an appreciable degree of expression and is relatively tumor-specific. Described herein is a method to transform heterogeneous RNA expression data in order to make meaningful comparisons across distinct data cohorts, sequencing protocols, and instruments.

Applicant's CAR-T prediction algorithm utilizes information regarding gene localization, transformed RNA expression values, and relative protein expression to assess a tumor-specific set of CAR-T targets. Applicant demonstrated the efficacy of the present method using clinically effective targets in B-cell Acute Lymphoblastic Leukemia. The methodology is extrapolated to predict effective CAR-T targets in pediatric Acute Myeloid Leukemia tumors of AMKL subtype given patient prognoses are poor. Applicant identified AML CAR-T targets currently undergoing clinical trials, as well as novel CAR-T targets in pediatric AMKL tumors.

### Results

### Data Transformation to Improve Heterogeneous Data Comparison

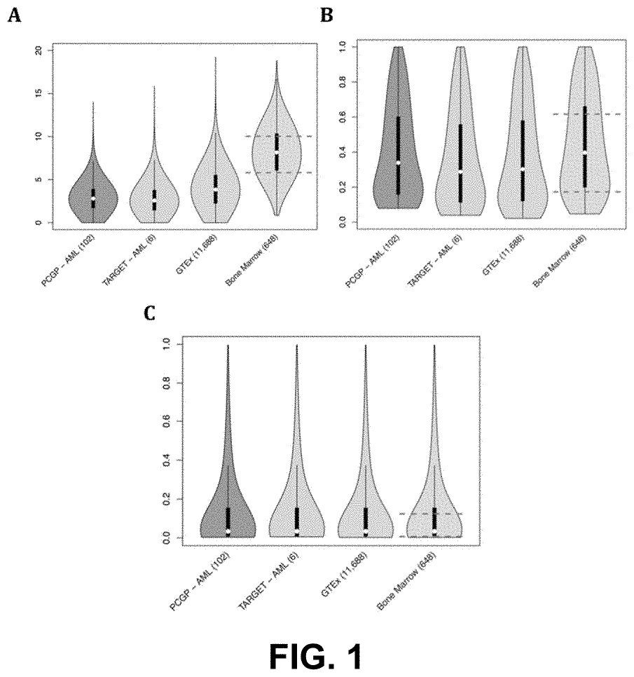

Representing gene expression values as fragments per kilobase of transcript per million mapped reads (FPKM) is a standard practice for RNA-seq data. FPKM values can vary significantly in their distribution between data cohorts. Microarray data also have their own unique distribution, which can make comparisons difficult across publicly available datasets (FIG. 1A).

Applicant performed a CUMULPERC to transform gene expression values in RNA-seq and microarray data to a range of values from [0,1] to indicate relative expression (FIG. 1B). Finally, the samples were normalized by quantile, using the transformed microarray gene expression values as the target distribution, to make the distribution of gene expression values equivalent between tumor and control samples (FIG. 1C). In doing so, transformed gene expression values, as well as microarray-derived gene status predictions—noted by definition of gene presence or absence—can be compared across samples, experiments, and data cohorts.

### Model-based Assessment of Gene Presence

The ability to compare RNA-seq and microarray data yields more than simply a comparison of the intensity of detected gene expression. Microarray data is unique from RNA-seq data in that the microarray data comes in two forms simultaneously: hybridization value intensities as well as an assessment of probable gene absence or presence in the sample^{19,20}. When RNA-seq and microarray data are transformed in a manner in which they can be meaningfully compared, additional information in the form of gene expression status prediction can be obtained from the microarray data and applied to RNA-seq values.

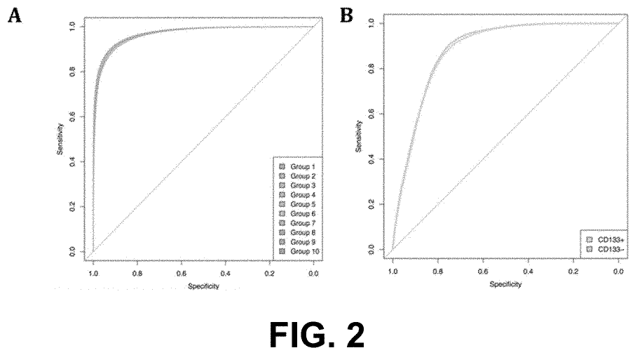

To assess the capability of a logistic regression model to predict gene presence and absence based solely on transformed hybridization value intensity in microarray data, a ten-fold cross-validation was performed on 648 bone marrow microarray samples²¹. Groups 1-9 were assigned 65 samples per group and Group 10 was assigned the remaining 63 samples. The logistic regression model predicted gene status in the ten-fold cross validation with 90.8±0.36% accuracy, 91.6±0.90% sensitivity, and 89.6±1.09% specificity relative to microarray gene status prediction based upon transformed probe hybridization values (FIG. 2A-B).

Further, a distinct set of six microarray samples from the Affymetrix Human Genome U133 Plus 2.0 platform were observed to assess the predictive ability for the logistic regression model across data cohorts. The samples were cell-sorted into CD133+ and CD133− cell populations²². The logistic regression model predicted gene status with 79.7±1.76% accuracy, 78.0±3.29% sensitivity, and 82.2±0.74% specificity from the transformed values in CD133+ cells. In CD133− cells, gene status was predicted with 79.4±0.79% accuracy, 76.7±0.31% sensitivity, and 83.3±1.69% specificity (FIG. 2C-D). The model correctly identified the CD133+ and CD133− subpopulations solely from the transformed hybridization value intensities.

The logistic regression model that was built upon transformed microarray expression values was then applied to the transformed FPKM values from pediatric B-ALL and AML patients. Common housekeeping genes, as well as those that are externally defined, were observed to assess the model's ability to predict expected gene presence. The logistic regression model was able to detect gene presence in housekeeping genes with 99.8% accuracy in the 304 B-ALL and AML pediatric patients observed. To assess the model's ability to predict expected absence of gene expression, tissue-specific antigens that are not associated with presence in B-ALL or AML were observed (Table 1).

When applying the logistic regression model to CAR candidates, gene presence data can be generated as an additional metric to calculate an estimated threshold of patients that have a particular antigen presence in tumor, and antigen absence in control tissue critical for regular biological function. Conversely, gene status information can exclude potential target antigens, or identify a set of patients that are not recommended for CAR-T therapy using a particular antigen, due to antigen absence in tumor or presence in critical control tissue.

### CAR-T Target Prediction Algorithm

Applicant developed an algorithm to identify potential CAR-T targets using microarray, RNA-seq, and protein expression data in tumor and control tissues. First, effective CAR-T targets must be present on the plasma membrane of the targeted cellular population in order for the CAR-T to recognize the specific cellular antigen presence necessary for binding and activation of the CAR-T co-stimulatory domains to induce cell lysis. Potential membrane association was assigned to genes via Gene Ontology (GO) terms specified in Ensembl and the Human Protein Reference Database.

Wilcoxon-Mann-Whitney tests were performed to identify genes with significantly greater expression in tumor relative to normal tissue³⁵. Potential CAR-T targets were initially ordered based on the degree of statistical significance. A threshold for RNA expression was set to only consider genes with a transformed expression value greater than 0.5 in primary tumor samples. In addition, a threshold was set for a maximum value of 0.3 in normal tissue from GTEx database. A minimum expression value threshold was not set in the bone marrow dataset, since clinically effective CAR-T targets, like CD19, can demonstrate a high expression values in bone marrow. High CD19 expression in the bone marrow is likely due to its known role in B cell development, but this phenomenon has not significantly limited the efficacy of the CD19 CAR-T target.

Protein expression is then considered using immunohistochemistry data from the Human Protein Atlas. CAR-T candidates were excluded as potential targets if protein expression was observed with an intensity of “low” or higher in more than 7 normal tissues. Germline tissues were excluded from this filter, since cancer-testis antigens have demonstrated expression in tumor resulting in efficacy as targets of immunotherapy³⁸⁻⁴⁰.

The remaining targets were then subject to manual review to confirm the candidates' potential efficacy as a CAR-T therapy. Extensive literature review and data mining further assessed the confidence associated with extracellular exposure. GO Experimental and High Throughput evidence codes superseded those that were solely based on computational analysis³². Gene expression value thresholds were observed to flag targets that may have too low expression to be targeted in tumors, or too high expression in critical, normal tissue to serve as effective CAR-T targets. CAR-T targets were also excluded if their co-localization on the plasma membrane was questionable.

### Performance Assessment of Effective CAR-T Targets in Pediatric B-ALL

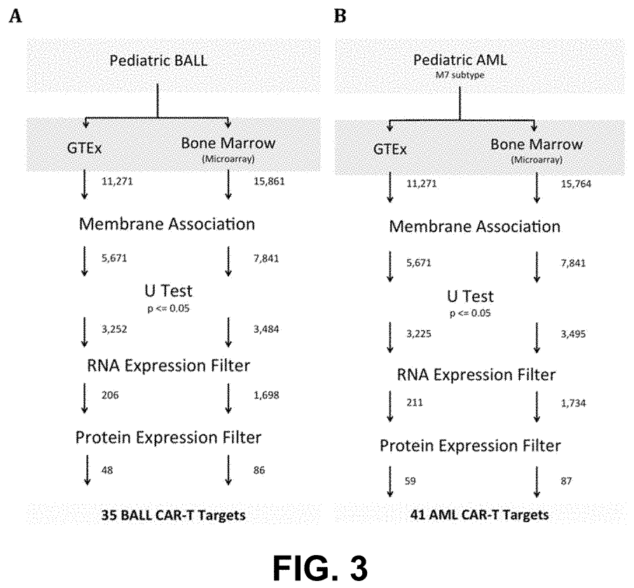

Successful CAR-T targets have been demonstrated in the treatment of B-cell malignancies like CD19 and CD22. Therefore, our CAR-T prediction algorithm was first applied to data derived from 196 pediatric B-ALL patients for performance assessment (FIG. 3A). Two CAR-T targets, CD19 and CD22, that have been effective in clinical practice in B-ALL patients were among the 28 CAR-T targets predicted by this algorithm in pediatric B-ALL. Several other known B cell surface markers—CD11a, CD38, CD45, CD69, CD72, CD79a, CD79b, CD132, and CD179b—were identified as potential CAR-T targets due to overexpression in B-ALL. The identification of clinically relevant CAR-T targets in B-ALL demonstrates the efficacy of our algorithm, which can be applied to other diseases (FIG. 3A).

### Novel CAR-T Target Prediction in Pediatric AML

Clinically effective CAR-T targets have yet to be shown in pediatric AML patients. Given that AML patients of AMKL subtype currently have a particularly poor prognosis, we focused on the identification of CAR-T targets in 108 AMKL patients in the PCGP and TARGET databases.

41 potential AMKL CAR-T targets were identified (FIG. 3B). The prediction of gene status for CAR-T targets identified by our algorithm in AMKL had similar profiles to clinically effective CAR-T targets in B-ALL (Table 2). Manual review of the 41 identified targets resulted in the removal of 14 genes due to known expression on vital tissue or subcellular localization that was not exposed to the cell's exterior. 15 of the remaining 27 AMKL CAR-T targets are members of the cluster of differentiation (CD) family. CD family genes indicate the capability for antigen recognition of the CAR-T targets via monoclonal antibody.

### DISCUSSION

Given the continuous increase in the number and diversity of public datasets, establishing a method that can make meaningful comparisons between datasets is critical. Having the capability to compare heterogeneous data presents an opportunity for further utility of experiments that have been performed and peer-reviewed. If reliably comparable, the use of heterogeneous public data could foreseeably be utilized to drive or support the conclusions of novel research.

Applicant has presented the difficulty of comparing gene expression values between data cohorts. To address this problem, Applicant has developed a data transformation method to make meaningful value-to-value comparisons between data cohorts and experiment types. Applicant has demonstrated the efficacy of the value transformation method described herein relative to other commonly used methods of value normalization regarding relative gene expression within a sample (FIG. 1).

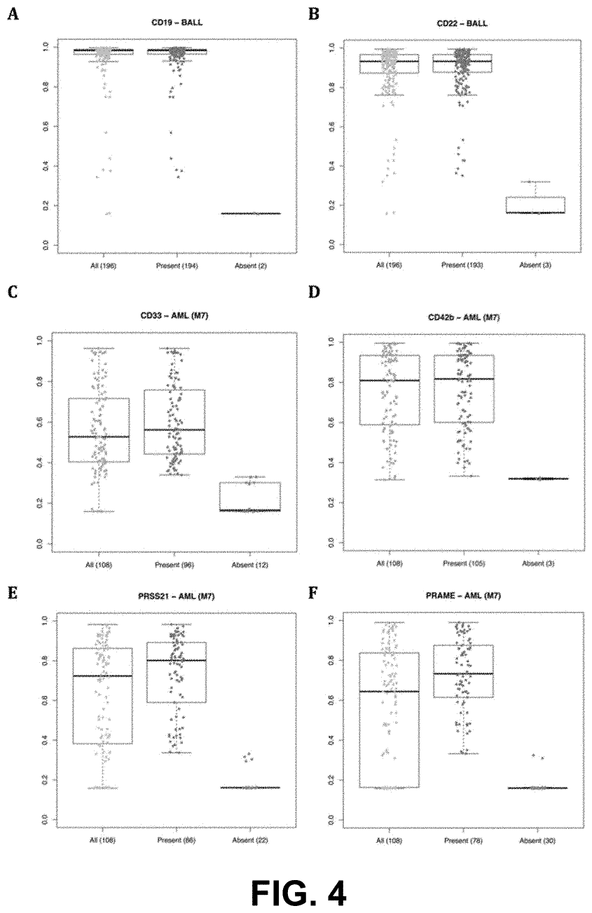

Beyond value-to-value expression comparisons, we have also shown the value of microarray gene status data as an effective metric to assess significant gene expression in RNA-seq data. Applicant has generated a logistic regression model, based upon transformed microarray hybridization values, to predict gene status strictly based upon transformed FPKM values (Table 1). The logistic regression model predicted gene status with relatively consistent accuracy, sensitivity, and specificity using microarray hybridization values in a ten-fold cross-validation, as well as an assessment in an external microarray dataset (FIG. 2). Applicant then applied the logistic regression model to infer gene status of CAR-T targets on a patient-by-patient basis using transformed RNA-seq expression values. Applicant's predicted AMKL CAR-T targets have a similarly high degree of gene expression in tumor and low gene presence in control samples as clinically effective CAR-T targets in B-ALL (Table 2). Further, Applicant has shown high expression of the top candidate CAR-T targets (FIG. 4). Identified AMKL CAR-T targets exhibited appreciable expression in at least 72% of the AMKL patients (Table 2). An objective assessment of sample-specific gene status as described herein—by utilizing microarray expression values and the corresponding prediction of gene presence or absence—could foreseeably be useful in the context of objective experimental condition confirmation, defining a meaningful threshold to filter out low gene expression before gene expression analysis, or potentially even inferring the patient cohort of targeted therapy.

Taken together, Applicant has demonstrated an application of transformation methods that can be used to compare gene expression value across data cohorts and even experiment types. Applicant assessed potentially therapeutic candidates for CAR-T cell therapy in B-ALL to confirm that our algorithm is effective at identifying clinically effective CAR-T targets like CD19 and CD22. Applicant then applied this algorithm to pediatric AMKL patient data—for which there is currently poor patient prognosis and no effective CAR-T treatment—to provide potentially effective CAR-T targets.

Most of the early-phase clinical trials for adult AML patients has focused on genes in the CD family. Our algorithm identified three genes currently in AML clinical trials—CD33, and CD38⁵¹. Another gene in AML clinical trials, CD32, was deemed to be a strong hit, but was excluded due to expression on 14 control tissues. It is important to note that clinical trials have been focused on adults rather than pediatrics.

55.6% of the CAR-T targets identified in our algorithm in pediatric AMKL patients were gene in the CD family. 2 CD family genes identified by our algorithm are currently undergoing adult clinical trials and 13 CD family genes identified represent novel potential as pediatric AMKL CAR-T targets.

Other genes that show particular promise as AMKL CAR-T targets in this data were PRSS21 and PRAME. We noted overexpression of the genes in AMKL primary tumor samples, and when considering control tissue, RNA and protein expression of the genes was almost exclusively limited to testis. Cancer-testis antigens have demonstrated anti-tumor efficacy. Previous research supports the notion that PRSS21 are PRAME are targetable antigens on the plasma membrane. Our algorithm also identified KCNN4 as a candidate for AMKL CAR-T therapy, which is a FDA approved drug target in sickle cell anemia.

### Materials and Methods

### Data Sources

RNA-seq data from was observed in 102 AML patients of AMKL subtype from previously published Pediatric Cancer Genome Project (PCGP) and 6 AML patients of AMKL subtype identified in the Therapeutically Applicable Research to Generate Effective Treatments (TARGET) data cohort.

To consider potential off-target effects of potential CAR-T targets, 11,688 RNA-seq samples were considered across 53 normal human tissues from the Genotype-Tissue Expression Project (GTEx). Also, 648 Affymetrix Human Genome U133 Plus 2.0 Array samples generated from the Microarray Innovations In Leukemia (MILE) study in human bone marrow tissue were also used as controls and in building the logistic regression model to predict gene presence in RNA-seq data. Six CD133 cell sorted microarray samples were used for an evaluation of the logistic regression model's performance when analyzing an external data.

### Expression Value Transformations

Gene expression value transformations were performed on the largest common gene subset amongst the data cohorts under comparison. In microarray expression data where multiple probes were associated with the same gene, the largest expression value was considered. The transformation methods described below can be applied to gene expression values across platforms that assess relative gene expression. Applicant has described the efficacy of the transformations in making more reliable comparisons between data cohorts, as well as between RNA-seq and microarray gene expression data.

### CUMULPERC

CUMULPERC is a finer discriminator of relative gene expression intensity within a sample relative to value binning. The CUMULPERC method transforms non-negative log 2 raw expression values of descending intensity into a continuous value whose range is 0-1 with 0 being the least possible expression assigned and 1 being the highest. The CUMULPERC value transformation method can be described as;

$${g}_{i}^{′} = 1 - \frac{{\sum }_{i}^{\# genes in sample} {g}_{i}}{Sample Total}$$

where gᵢ represents the cumulative non-negative log 2 raw expression value and g′ᵢ, represents the transformed value calculated at each iteration. Genes with the same non-negative log 2 raw expression value are assigned the same transformed value without increasing

### Data Analysis

The data analysis and visualization described herein was conducted using self-developed bash and R scripts. The BiocParallel, MASS, pheatmap, and plyr R packages was used within the script for parallel processing, logistic regression modeling, visualization, and object manipulation, respectively.

TABLE 1
Modeling of Gene Status Using Normalized Gene Expression Values.
The presence or absence of gene expression was predicted using
a model built from transformed microarray values and their associated
gene status predictions. Commonly observed and empirically defined
housekeeping genes were observed to demonstrate the capability
of the model to accurately detect gene presence solely using
transformed RNA-seq expression values. Gene absence was assessed
by observing genes that are tissue-specific and not expected
to be present in B-ALL nor AML.

| | BALL | AML (AMKL) | Control |
|---|---|---|---|
| | % Present | % Present | % Present |
| Housekeeping Genes | | | |
| GAPDH | 100% | 100% | 100% |
| ACTB | 100% | 100% | 100% |
| CHMP2A | 100% | 100% | 100% |
| EMC7 | 100% | 100% | 100% |
| GPI | 100% | 100% | 100% |
| PSMB2 | 100% | 100% | 100% |
| PSMB4 | 100% | 100% | 100% |
| RAB7A | 100% | 100% | 100% |
| REEP5 | 100% | 99% | 100% |
| Tissue-specific Antigens | | | |
| (Non-BALL and Non-AML) | | | |
| EGFR^{23,24} | 0% | 0% | 95% |
| MUC16²⁵ | 0% | 1% | 9% |
| CEACAM5^{26,27} | 0% | 0% | 7% |

TABLE 2
CAR-T Candidate Gene Expression Status Prediction in RNA-seq Data.
Results of the logistic regression model gene status prediction across
AML patients' transformed FPKM values. Housekeeping genes were
defined by common use in the scientific community as well as those
that were objectively defined. Tissue-specific antigens represent
previously identified antigens specific to non-AML cancers. Gene
status assessment of the CAR-T targets may be indicative of the percent
of the observed population that may or may not be candidates for
CAR-T cell treatment using a specific antigen. CD32 has an asterisk
to indicate that it was not a bonafide hit in Applicant's algorithm
due to levels of protein expression in control tissue.

| BALL | BALL | Control |
|---|---|---|
| CAR Targets | % Present | % Present |
| CD19 | 99% | 4% |
| CD22 | 99% | 28% |

| AML (AMKL) | AML (AMKL) | Control |
|---|---|---|
| CAR Targets | % Present | % Present |
| CD33 | 89% | 7% |
| CD38 | 87% | 12% |
| GP1BA (CD42b) | 97% | 2% |
| CD69 | 100% | 11% |
| CD83 | 100% | 9% |
| CTSW | 100% | 5% |
| KCNN4 | 99% | 18% |
| ITGA2B (CD41) | 100% | 20% |
| PTPRCAP (CD45) | 100% | 7% |
| GPR174 | 90% | 1% |
| RASAL3 | 100% | 11% |
| GP6 | 80% | 2% |
| GP9 (CD42a) | 94% | 5% |
| ABCC4 | 100% | 17% |
| GYPA (CD235a) | 77% | 2% |
| IL2RG (CD132) | 100% | 29% |
| CD96 | 80% | 5% |
| PRSS21 | 80% | 5% |
| ITGB3 (CD61) | 94% | 28% |
| CD7 | 85% | 8% |
| AGER | 99% | 18% |
| CARD11 | 88% | 10% |
| MS4A2 (CD20) | 72% | 4% |
| PRAME | 72% | 7% |
| MLC1 | 100% | 40% |
| CMTM5 | 86% | 31% |
| ITGAX (CD11c) | 88% | 32% |
| FCGR2A (CD32)* | 94% | 15% |

The disclosure of every patent, patent application, and publication cited herein is hereby incorporated herein by reference in its entirety.

While this invention has been disclosed with reference to particular embodiments, it is apparent that other embodiments and variations of this invention can be devised by others skilled in the art without departing from the true spirit and scope of the invention. The appended claims include all such embodiments and equivalent variations.

What is claimed is:

1. A method for diagnosing and/or prognosing a cancer associated with PRSS21 expression in a patient, the method comprising:
measuring the level of PRSS21 in a biological sample obtained from the patient by contacting the biological sample with a PRSS21 detection agent, and measuring the absence, presence or expression level of an expression product of PRSS21, wherein increased level of the PRSS21 expression product compared to control indicates that the patient has cancer associated with PRSS21 expression.

2. The method of claim 1, wherein the cancer is selected from the group consisting of: a leukemia, B-cell acute lymphoblastic leukemia (B-ALL), acute myeloid leukemia (AML) pediatric B-ALL, pediatric AML, AML of the AMKL subtype, and a solid tumor.

3. The method of claim 1, wherein the biological sample is selected from the group consisting of: fluids (e.g., blood, plasma, serum, bone marrow aspirate, bronchoalveolar lavage (BAL), cerebrospinal fluid (CSF)), tissues, cell samples, organs, biopsies, or tumor samples.

4. The method of claim 1, wherein the expression product comprises an RNA, mRNA, a protein, or a fragment thereof.

5. The method of claim 1, wherein the detection agent comprises a nucleic acid (DNA or RNA probe) or an antibody.

6. The method of claim 1, wherein the expression is measured by array hybridization, direct hybridization of RNA, digital quantitation of transcript levels, quantitative PCR, quantitative sequencing, northern blot analysis, mass spectrometry, ELISA, flow cytometry, immunohistochemistry, radioimmunoassay, western blot, or immunoprecipitation.

7. A method of identifying a subject having cancer as a candidate for an anti-cancer immunotherapy, said method comprising determining an expression level of PRSS21 in a biological sample obtained from the subject, wherein an increased expression level of PRSS21 in the biological sample from the subject compared to a reference expression level in a non-cancerous sample is indicative of the subject being a candidate for anti-PRSS21 immunotherapy.

8. The method of claim 7, wherein the cancer is selected from the group consisting of: a leukemia, B-cell acute lymphoblastic leukemia (B-ALL), acute myeloid leukemia (AML) pediatric B-ALL, pediatric AML, AML of the AMKL subtype, and a solid tumor.

9. A method of identifying a subject as a candidate for an anti-cancer immunotherapy comprising a chimeric antigen receptor (CAR)-T cell therapy, said method comprising determining an expression level of PRSS21 in a biological sample obtained from the subject, wherein an increased expression level of PRSS21 in the biological sample obtained from the subject compared to a reference expression level in a non-cancerous sample is indicative of the subject being a candidate for anti-PRSS21 immunotherapy.

10. A method of identifying a subject suffering from acute myeloid leukemia (AML) or a solid tumor as a candidate for an anti-cancer immunotherapy comprising a chimeric antigen receptor (CAR)-T cell therapy, said method comprising determining an expression level of PRSS21 in a biological sample obtained from the subject, wherein an increased expression level of PRSS21 in the biological sample obtained from the subject compared to a reference expression level in a non-cancerous sample is indicative of the subject being a candidate for anti-PRSS21 immunotherapy.

11. A method of treating a subject having a cancer associated with an elevated expression of protease serine 21 (PRSS21), said method comprising administering to the subject an effective amount of an immunotherapeutic composition comprising an anti-PRSS21 antibody or an immune effector cell targeted to PRSS21.

12. A method of treating a subject suffering from acute myeloid leukemia (AML) or a solid tumor associated with an elevated expression of protease serine 21 (PRSS21), said method comprising administering to the subject an effective amount of an immunotherapeutic composition comprising an anti-PRSS21 antibody or an immune effector cell targeted to PRSS21.

13. A method for targeted anti-cancer immunotherapy in a subject suffering from acute myeloid leukemia (AML) or a solid tumor associated with an elevated expression of protease serine 21 (PRSS21), wherein the anti-cancer immunotherapy comprises administration of a therapeutically effective amount of a CAR-T cell.

14. The method of claim 13, wherein the AML comprises an AMKL subtype.

15. A method for treating a subject with an anti-PRSS21 immunotherapy, wherein the subject is suffering from a PRSS21-associated disorder, the method comprising the steps of: measuring the level of PRSS21 in a biological sample obtained from the patient by contacting the biological sample with a PRSS21 detection agent, and measuring the absence, presence, or expression level of an expression product of PRSS21, and administering a therapeutically effectively amount of an immunotherapeutic agent to the patient upon measuring increased levels of the PRSS21 expression product compared to a proper control.

16. The method of claim 15, wherein the PRSS21-associated disorder comprises a cancer.

17. The method of claim 16, wherein the cancer is selected from the group consisting of: a leukemia, B-cell acute lymphoblastic leukemia (B-ALL), acute myeloid leukemia (AML), pediatric B-ALL, pediatric AML, AML of the AMKL subtype, and a solid tumor.

18. The method of claim 16, wherein the cancer comprises AML of the AMKL subtype.

19. The method of claim 15, wherein PRSS21 level is measured by array hybridization, direct hybridization of RNA, digital quantitation of transcript levels, quantitative PCR, quantitative sequencing, northern blot analysis, mass spectrometry, ELISA, flow cytometry, immunohistochemistry, radioimmunoassay, western blot, or immunoprecipitation.

20. The method of claim 15, wherein the immunotherapeutic agent comprises a CAR-T.
